(19) United States
(12) Patent Application Publication
(10) Pub. No.: US 2026/0177710 A1
(43) Pub. Date: Jun. 25, 2026

(54) IMPROVED TIME-OF-FLIGHT X-RAY IMAGING USING TPSF

(71) Applicant: SOCPRA SCIENCES ET GÉNIE S.E.C., Sherbrooke (CA)

(72) Inventors: Réjean FONTAINE, Sherbrooke (CA); Julien ROSSIGNOL, Sherbrooke (CA); Yves BÉRUBÉ-LAUZIÈRE, Sherbrooke (CA)

(21) Appl. No.: 19/125,183

(22) PCT Filed: Nov. 3, 2023
(86) PCT No.: PCT/CA2023/051475

§ 371 Date: Apr. 28, 2025

Related U.S. Application Data
(60) Provisional application No. 63/422,636, filed on Nov. 4, 2022.

(51) Int. Cl. G01T 1/29; A61B 6/03; A61B 6/40; G01N 23/046

(52) U.S. Cl. CPC G01T 1/2914; A61B 6/032; A61B 6/4085; G01N 23/046; G01N 2223/045

(57) ABSTRACT

Providing an X-ray measurement with a reduction in the measure of scattered photons is significantly advantageous over measurements with a usual contribution of scattered photons. Using a temporal point spread function (TPSF) of the detected photons, for each source-detector location, to discriminate the scattered photons from the ballistic photons is used to significantly relax the temporal resolution requirement at the time-sensitive X-ray detector of the X-ray imager when compared to the low temporal resolution required when the discrimination is carried out on a photon-by-photon basis. Using the TPSF dataset provides better estimates of the measure of ballistic photons with a reduction in the measure of scattered photons which are also used to generate X-ray images with an improvement in CNR.

### CROSS-REFERENCE TO RELATED APPLICATIONS

This application claims priority from U.S. provisional patent application 63/422,636 filed Nov. 4, 2022, the specification of which is hereby incorporated by reference.

### TECHNICAL FIELD

This present patent application relates to X-ray imaging.

### BACKGROUND

In X-ray imaging, X-photons from a source are transmitted through a target object to a detector. A reduction in detectable X-photons is related to absorption in the target object, and an image is generated from detected X-photons. However, both ballistic and scattered photons are detected. The ratio between ballistic and scattered photons depends on the energy of the X-photons, the thickness/density of the target object being imaged, the size of the irradiated volume and the detector solid angle. Normally, the proportion of scattered X-photons is small enough to be ignored in conventional helical computed tomography. However, in X-ray radiography and cone-beam computed tomography, it is significant enough to degrade the contrast-to-noise ratio (CNR).

In international patent application (PCT) publication WO 2020/093140 A1, published on May 14, 2020, an X-ray imaging system is disclosed that uses a pulsed X-ray source and a time-sensitive detector that is able to discriminate between ballistic and scattered X-photons by their time of flight. Images based on a reduced measure of scattered X-photons were shown to have an improved CNR.

Obtaining a real-time measurement of the proportion of scattered photons while receiving simultaneously both scattered and ballistic photons is a difficult task. Since usual X-ray sources for X-ray imaging use a large energy range, from 20 keV to 120 keV, the energy of the individual photons cannot be used to measure how many have been scattered in between the source to the detector.

Measuring the time-of-flight requires some adjustments to the conventional X-ray imaging system. Namely, the X-ray must be sent in short pulses instead of continuously and the detector must be time-resolved. As scattered photons travel for a longer time from the source to the detector than ballistic photons, this scheme can be used to identify scattered photons and remove them directly. However, this approach requires ultrafast detectors to remove most of the scattered photons from the measurements, since the difference of time-of-flight between a scattered and a ballistic photon is often below 100 picoseconds. A similar or better time resolution of the time-of-flight of the detected photons is needed to accurately distinguish and discriminate between ballistic and non-ballistic photons. This may severely hinder the development of imaging systems using this kind of measurement for scatter removal since they may be unaffordable or unavailable.

A precise X-ray detector must be dense enough to stop X-rays reliably within a short distance and must generate an electric signal with low time jitter. Semiconductor detectors and scintillators are often used for X-rays detection. Semiconductor detectors directly convert X-rays to electric signals but offer poor time precision. Scintillators have been the standard for high-precision photon detection for a few decades in positron emission tomography, another medical imaging modality. These materials emit visible light when X-rays interacts in them, which then can be measured using a photodetector such as a photodiode or a photomultiplier tube. Timing-jitter of less than 100 picoseconds is possible for gamma rays and soft (<10 kev) X-rays using scintillators. It is, however, challenging to be as precise in the medical X-ray energy range. Most precise scintillators are either not dense enough for 20-120 keV X-rays or do not generate enough light at this energy to reliably be detected by the photodetector. This, in turn, causes imprecision in the measurement. It is possible to circumvent the density problem by increasing the length of the detector. However, long detectors cause significant timing jitter since the position of interaction of the X-rays in the scintillator has more variation, thus, the visible light reaches the photodetector with a similar variation. Because of these challenges, reaching the high time resolution required for time-of-flight scatter removal is difficult and would significantly increase the cost of an X-ray imaging apparatus using this technique.

A solution to this problem by significantly relaxing the timing requirement of detectors for time-of-flight based scatter removal would be a useful and significant improvement.

### SUMMARY

Moving away from a time-of-flight X-photon discrimination on a photon-by-photon basis to remove scattered photons from the measurements to keep only ballistic photons, it was discovered that measuring the elapsed time from emission to detection of X-photons (time-of-flight), leading to a time-of-flight distribution of all X-photons, also known as a temporal point spread function (TPSF), for each source-detector location, relaxes the temporal resolution requirement at the time-sensitive detector and avoids the need for it to be lower than 20 picoseconds. This can allow to use time-sensitive detectors with a higher temporal resolution while still providing and generating high quality X-ray images (e.g., of same or better quality). This approach involves the acquisition of a statistically valid population of detected X-photon to form a TPSF dataset, which can be achieved efficiently over a large number of X-ray source pulses. This approach can tolerate a greater time variability in an X-ray source's pulses and in an X-ray detector's time resolution while still providing effective elimination of measured scattered X-photons, and thus leading to an improvement in CNR.

A broad aspect of the present disclosure is an X-ray imaging apparatus comprising a pulsed X-ray source having a control signal, a time-sensitive X-ray detector having a time-dependent X-ray photon detection signal output, and circuitry, connected to the control signal and the time-dependent X-ray photon detection signal output, for collecting a TPSF dataset from a time-dependent X-ray photon detection signal of the time-sensitive X-ray detector.

In some embodiments, the proposed X-ray imaging apparatus further comprises a processor and memory storing program code that, when executed by the processor, causes the processor to estimate a number of ballistic photons based on at least one shape parameter of a TPSF of the TPSF dataset to provide a measure of ballistic photons with a reduction in the measure of scattered photons.

In some embodiments of the proposed X-ray imaging apparatus, the estimation of the number of ballistic photons is based on a distribution resulting from a temporal deconvolution of the TPSF by an instrument response function of the X-ray imaging apparatus.

In some embodiments, the circuitry comprises the processor and the memory, and wherein the storing program code, when executed by the processor, causes the processor to perform the collecting of the TPSF dataset from the time-dependent X-ray photon detection signal.

In some embodiments, the proposed X-ray imaging apparatus further comprises an image processor that, when executing the program code stored in the memory, causes the image processor to generate an X-ray image from the measure of ballistic photons with a reduction in the measure of scattered photons.

In some embodiments of the proposed X-ray imaging apparatus, a temporal resolution of the time-sensitive detector is more than 20 picoseconds and less than about 300 picoseconds.

In some embodiments of the proposed X-ray imaging apparatus, the pulsed X-ray source comprises a high-voltage source, electrodes connected to the high-voltage source for accelerating electrons, and an X-ray emitting target material arranged to receive the electrons following acceleration by the electrodes so as to produce a pulse of X-rays.

In some embodiments of the proposed X-ray imaging apparatus, the pulsed X-ray source comprises a pulsed laser source responsive to the control signal, a photoelectric material arranged to receive a light pulse from the pulsed laser source and to emit a burst of electrons in response thereto, wherein electrodes are arranged to accelerate the burst of electrons. In some of these embodiments, the photoelectric material is at least a part of a cathode of the electrodes.

In some embodiments of the proposed X-ray imaging apparatus, the pulsed X-ray source comprises deflection electrodes for steering the electrons accelerated by the electrodes connected to the high-voltage source to controllably hit the X-ray emitting target material.

In some embodiments of the proposed X-ray imaging apparatus, the electrodes connected to the high-voltage source comprises a gated carbon nanotube cathode.

In some embodiments of the proposed X-ray imaging apparatus, the time-sensitive X-ray detector is arranged with respect to the pulsed X-ray source so as to provide a different time of flight for the ballistic photons as a function of pixel location within the time-sensitive X-ray detector.

In some embodiments of the proposed X-ray imaging apparatus, the time-sensitive X-ray detector comprises an X-ray sensitive scintillator and a light sensor array coupled to the scintillator for measuring X-ray detection events in the scintillator.

In some embodiments of the proposed X-ray imaging apparatus, the time-sensitive X-ray detector comprises an X-ray sensitive detector based on a direct conversion of photons to electrons for measuring X-ray events in the detector.

In some embodiments of the proposed X-ray imaging apparatus, wherein, when the image processor executes the program code, causes the image processor to provide a two-dimensional image.

In some embodiments, the proposed X-ray imaging apparatus further comprises a motorized mounting for moving the pulsed X-ray source and the time-sensitive X-ray detector with respect to an object or subject to be imaged, wherein, wherein, when the image processor executes the program code, causes the image processor to provide a three-dimensional image.

In some embodiments of the proposed X-ray imaging apparatus, the pulsed X-ray source produces a cone beam and the time-sensitive X-ray detector is arranged in a 2D array of pixels to detect photons.

In some embodiments of the proposed X-ray imaging apparatus, a temporal uncertainty between the time of the control signal and detection of an X-photon by the time-sensitive X-ray detector is greater than 200 picoseconds.

In some of these embodiments, an image generated from the measure of ballistic photons with a reduction of scattered photons has a contrast-to-noise ratio at least 8% higher than the contrast-to-noise ratio of an image generated from a measure of ballistic photons with a reduction of scattered photons having a time-of-flight higher than a predetermined time-of-flight.

Another broad aspect of the present disclosure is an apparatus for producing an X-ray image comprising a processor, and memory storing program code, wherein the program code, when executed by the processor, causes the processor to estimate, for each TPSF of a provided TPSF dataset, a number of ballistic photons based on at least one shape parameter of the TPSF to provide a measure of ballistic photons with a reduction in the measure of scattered photons, and generating an image from the measure of ballistic photons with a reduction of measured scattered photons.

Some embodiments of the apparatus for producing an X-ray image further comprise a computed tomography image processor and wherein said program code, when executed by said computed tomography image processor, causes said computed tomography image processor to generate an X-ray image from each of the measure of ballistic photons with a reduction in the measure of scattered photons.

In some embodiments of the apparatus for producing an X-ray image, the computed tomography image processor is the processor used for the estimating the number of ballistic photons.

In some embodiments of the apparatus for producing an X-ray image, the estimation of the number of ballistic photons is based on a distribution resulting from a temporal deconvolution of the TPSF by an instrument response function of the X-ray imaging apparatus.

Yet another broad aspect of the present disclosure is a method for generating an X-ray image comprising: providing an X-ray imaging apparatus comprising a pulsed X-ray source and a time-sensitive X-ray detector; collecting a TPSF dataset from a time-dependent X-ray photon detection pixel signal of the time-sensitive X-ray detector, wherein the TPSF dataset comprises a TPSF for each one of a plurality of detector cells of the time-sensitive X-ray detector, estimating the number of ballistic photons based on at least one shape parameter of one of a plurality of the TPSF comprised in the TPSF dataset to provide a measure of ballistic photons with a reduction in the measure of scattered photons, providing an image processor, and generating, with the image processor, an X-ray image from the measure of ballistic photons with a reduction in the measure of scattered photons.

In some embodiments, the proposed method can collect a TPSF dataset for multiple signals (a time-dependent X-ray photon detection signal for each pixel of the time-sensitive X-ray detector) and can estimate the number of ballistic photons for each one of the TPSF datasets.

### BRIEF DESCRIPTION OF THE DRAWINGS

The invention will be better understood by way of the following detailed description of embodiments of the invention with reference to the appended drawings, in which:

FIG. 1 presents an embodiment of a detector comprising a physical anti-scatter filter.

FIG. 2A illustrates an embodiment of a two-dimensional (fan-shaped beam) computed tomography imaging apparatus.

FIG. 2B illustrates differences of time-of-flight, for a given pixel of the detector, between ballistic photons may be associated with the effect of spatial uncertainty on the exact location of emission and detection.

FIG. 3A shows an exemplary ideal time-of-flight distribution delivered by an X-ray imaging system that comprises a temporal distribution of the ballistic photons (peak) and of the scattered photons (tail) detected by one pixel of the detector.

FIG. 3B presents an embodiment of an empirical (“raw”) time-of-flight distribution (TPSF), where uncertainty sources have widened the ideal time-of-flight distribution of FIG. 3A.

FIG. 4A presents a block diagram of an embodiment of the proposed method.

FIG. 4B presents a block diagram of another embodiment of the proposed method.

FIG. 5A shows a simulation image with both ballistic and scattered photons considered and obtained with a simulation of regular (without time-of-flight filtering) prior art X-ray imaging (without filtering or jitter).

FIG. 5B shows a simulation image with only ballistic photons (ideal image without jitter).

FIG. 6A shows a simulation of a scatter corrected image using the proposed deconvolution with an applied 300 picoseconds timing jitter.

FIG. 6B shows a simulation of a scatter rejection (prior art) based on time-of-flight-photon discrimination on a photon-by-photon basis with an applied 300 picoseconds timing jitter.

FIG. 6C shows a simulation of a scatter corrected image using the proposed deconvolution method with an applied 100 picoseconds timing jitter.

FIG. 7 shows a schematic representation of an embodiment of the X-ray imaging apparatus.

FIG. 8A is an exemplary TPSF of only ballistic photons measured experimentally with nothing between the source and the detectors according to the time between the source trigger and the detection fitted with a landau distribution.

FIG. 8B is an exemplary TPSF measured experimentally with a 4 cm thick beam-blocker between the source and the detectors according to the time between the source trigger and the detection fitted with a landau distribution.

FIG. 9A is a schematic sectional side view of a laser-pulsed cathode side-window type of X-ray tube.

FIG. 9B is a schematic sectional side view of a carbon nanotube gated cathode side-window type of pulsed X-ray tube.

### DETAILED DESCRIPTION

In this patent, the term “object” is understood to mean an imaging phantom, a volume of interest, a body, part of a body, including bones, muscles, fat, organs and blood vessels, any object semi-permeable to or interacting with X photons, etc.

The following is a detailed description of embodiments of the disclosure depicted in the accompanying drawings. The embodiments are in such detail as to clearly communicate the disclosure without limiting the anticipated variations of the possible embodiments and may encompass all modifications, equivalents, combinations and alternatives falling within the spirit and scope of the present disclosure. It will be appreciated by those skilled in the art that well-known methods, procedures, physical processes and components may not have been described in detail in the following so as not to obscure the specific details of the disclosed invention.

The present invention provides a solution to the problem of detector performance and makes it possible to optimize performance (close to optimal performance) with detectors that are already available on the market. The solution can be significantly advantageous with respect to the development of new detectors by its simplicity and by the fact that it is mathematical and therefore may not necessarily requires the addition of hardware in the imaging apparatus. In the present disclosure, it will be appreciated that the proposed method can be relatively inexpensive to implement, has not been used in the field of time-of-flight CT and is totally new for this field.

It will be appreciated that the proposed method can also be implemented in an embedded processing system generating the temporal point spread function (TPSF), which can estimate the ballistic contribution from the TPSF directly or send it to another digital processor (embedded or not). In these embodiments, a modification or addition to the hardware can be required. Any computing device, e.g., GPUs, CPUs, FPGAs or digital ASICs, may be used to compute the estimation of a contribution of ballistic photons.

In computed tomography imaging, the prior art includes some X-ray imaging system and methods that can produce improved absorption images by essentially selectively removing the scattered X-photons measured during the acquisition of the X-photons so as to keep and use the ballistic photons reaching the detectors that are necessary to produce the absorption image (see PCT publication WO 2020/093140 A1). These techniques can use the measured time-of-flight (elapsed time from emission to detection) for each detected photon to discriminate between the ballistic photons, having a shorter time-of-flight (ToF), and the scattered photons having a longer ToF. In other words, a decision can be made on each individual detected X-photon (on a photon-by-photon basis) to identify and select those with a ToF shorter than a selected discrimination threshold (ToF expected for scattered photons). Such X-ray imaging system and methods can effectively produce accurate absorption images without requiring a specific configuration allowing to directly (e.g., during the acquisition) block non-ballistic photons, generally referred to as scatter rejection, by reducing the scatter contribution before it reaches the detector generally with collimators or through the imaging apparatus geometry.

In the context of the present disclosure or when interpreting the described method and apparatus, it will be appreciated that the concept, definition or value of a ToF can encompass, be transposed to or translated into equivalent time values (e.g., absolute or relative times, time of detection, timestamp). Therefore, through the current disclosure the term ToF may not be limited to the strict definition of a specific value of elapsed time between the emission of a X-photon at a source and its detection at a detector. For example, the ToF can encompass a relative time of detection (e.g., a time relative to or elapsed from an activation of the detector), the concept being that, although the monitored or known information may not be detailed enough to do so (e.g., to avoid/reduce some measurement or time resolution requirements), the considered value of time could theoretically be used to determine or calculate the true ToF (i.e., the value of elapsed time between emission and detection) if additional information was available (e.g., the time between the releasing of the source pulse and the activation of the detector). For example, a detection time may be converted to a ToF using a lookup table or known conversion factor or added correction value.

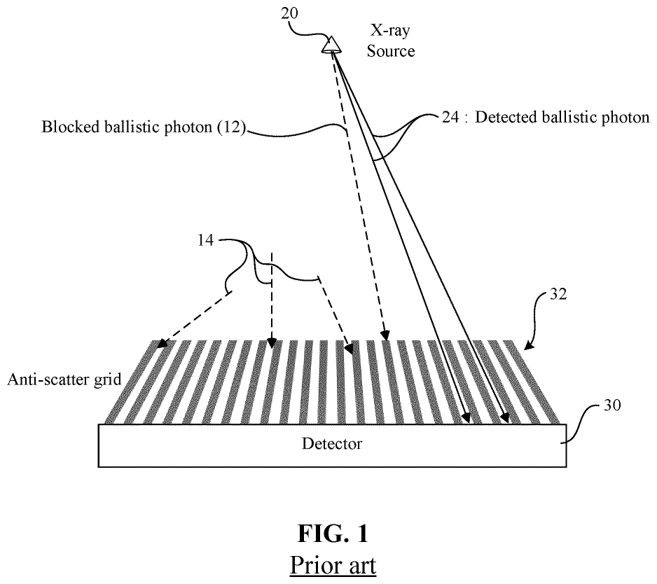

FIG. 1 presents an embodiment of a detector 30 comprising an alternative physical anti-scatter filter that was used in the prior art to physically block the non-ballistic photons 14 (e.g., scattered X-photons, ambient photons and others). Such an anti-scatter grid 32 may be added to the detector 30 in order to mostly limit the detection of photons to ballistic photons 24, but it will be appreciated that it also blocks a portion of the incident ballistic photons (i.e., blocked ballistic photons 12), up to about 30% in some cases, which may lead to an increase in noise in the resulting absorption images. Imaging apparatuses that use such anti-scatter filters may therefore require an increase in the number of X-photons emitted by the source to increase the number of detected X-photons (ballistic photons) by that same amount, up to about 30%, either by increasing the radiation concentration at the X-ray source 20 or by increasing the duration of the radiation exposure. This compensation (increase of emitted X-photons) inevitably leads to a higher radiation exposure of the patient, up to about 30% in some cases, which may limit the number scans or total amount of X-ray dose the patient can be submitted to for a given period (e.g., consecutive, day, week, month, etc.), possibly affecting the associated treatments, or, in extreme cases, may lead to health complications.

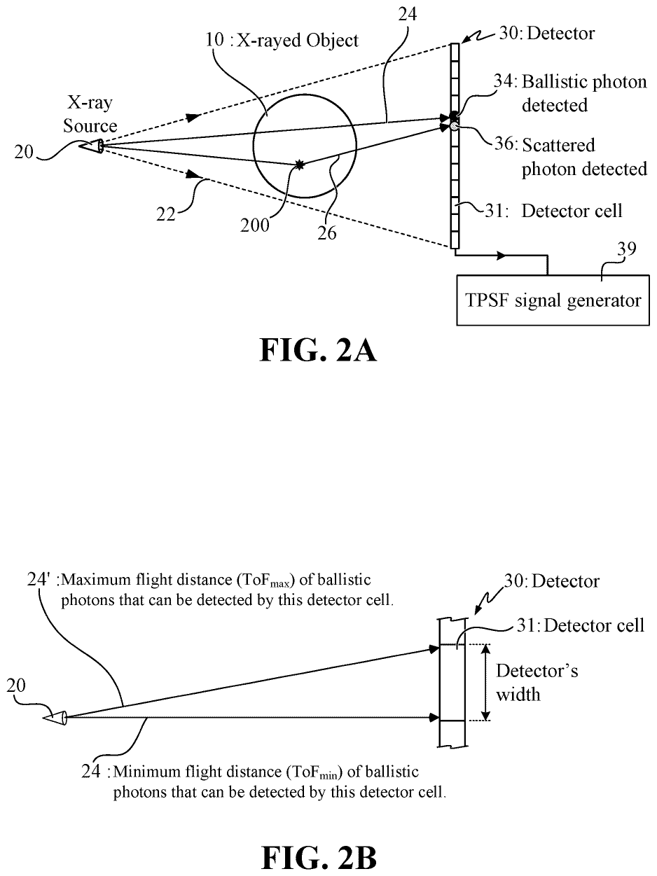

FIG. 2A illustrates a top view of a two-dimensional computed tomography imaging apparatus having a pulsed X-ray source 20 generating a 2D fan-shaped (or 3D cone-shaped) beam 22 that can send a pulse of X photons to an object 10 and towards a detector 30, here a one-dimensional detector array (a line of detector cells). A detector cell 31 can be a single photodetector subsection comprised in the detector array 30, which can have a small surface area and an associated position within the detector array 30. The detector cell 31 may be considered to be, and is referred herein as, a “pixel” of the detector array 30. The detector array may be comprised in and be part of a time-correlated single photon counting system that can be used to identify and eliminate scattered photons based on their ToF between the source and the detector, since scattered photons inherently have a longer ToF than ballistic photons. Here a 2D beam and a 1D detector are considered to simplify the description of the fundamental principles and dynamics on which the proposed method is based. This schematic representation further shows two types of the incident X photon that may be considered by the proposed method and apparatus. FIG. 2A shows a ballistic photon 34 having a direct flight path 24 that is typical if non-absorbed nor scattered photons and a once-scattered photon 36 (a photon scattered only once at the scatter position/event 200) having a deviated flight path 26. In this example, both photons originating from the source 20 (origin) at the same time, are detected at a slightly different position (destination) on the same detector cell 31 of the detector array 30 and therefore at different times (i.e., both having different times-of-flights). This specific case will help to appreciate that, since both ballistic and scattered photons have the same velocity, the total length of the deviated flight path 26 must be at least ever so slightly longer than the direct flight path 24 and may therefore have a longer ToF.

In an embodiment, the source 20 can be a pulsed X-ray source comprising a high-voltage source, electrodes connected to the high-voltage source for accelerating electrons, and an X-ray emitting target material arranged to receive the electrons following acceleration by the electrodes so as to produce a pulse of X-rays.

In an embodiment, the source 20 can be a pulsed X-ray source comprising: a pulsed laser source responsive to a control signal; a photoelectric material arranged to receive a light pulse from the pulsed laser source and to emit a burst of electrons in response thereto; wherein electrodes are arranged to accelerate the burst of electrons. In a non-limiting embodiment, the photoelectric material can at least a part of a cathode of the electrodes.

In an embodiment, the source 20 can be a pulsed X-ray source that can comprises deflection electrodes for steering the electrons accelerated by the electrodes connected to the high-voltage source to controllably hit the X-ray emitting target material.

The X-ray imaging apparatus can be equipped with TPSF signal generator 39 for processing the detection signal of the detector that can store, in memory, the measurement data and may do so separately for each pixel of the detector 30 (each detector cell 31) for each acquisition. Here, the TPSF signal generator 39 can comprise circuitry which may include: a field programmable gate array (FPGA), logic gates, integrated circuitry, processors (e.g., central processing unit), any suitable memory that may comprise/store instructions for properly controlling/driving these last components, if required, or any combination thereof. The TPSF signal generator 39 can comprise circuitry or a program for converting the various signals analog or digital (e.g., trigger signal of the X-ray source, photon detection signals of the detector's pixels, etc.) to a digital value (e.g., ToF of detected photons, timestamps, counts, positions, etc.) which may be stored into a database and/or converted into TPSF signals and/or a TPSF dataset. To do so, the TPSF signal generator 39 can comprise, for example, a time-to-digital converter (TDC) which can be, but is not limited to, the embodiments of a TDC described in the PCT application WO2021243451A1. In some embodiments, the generated digital values can be stored as a measured timestamp data which can comprise a timestamp matrix collecting, for each position of the various detector cell 31, each of the measured ToFs (timestamps of the photons detected by each detector cells 31).

The TPSF signal generator 39 can use the measured timestamp data to generate TPSF signals and/or a TPSF dataset by counting the number of detected photons associated with various timestamps for each detector's pixels to generate a corresponding histogram (e.g., TPSF).

In some embodiments the TPSF signal generator 39 can generate the TPSF of the acquisitions in real-time (e.g., as the photons are detected and/or from a time-dependent X-ray photon detection signal of the time-sensitive X-ray detector 30) or from the stored measurement data and can store the TPSF, in memory.

In some embodiments, this TPSF signal generator 39 can further comprise a program stored in memory (internal or external) containing instructions to perform the method proposed herein. In some embodiments, the TPSF signal generator 39 to process the detector's signal can perform one to all steps of the proposed method. The TPSF signal generator 39 can output a generated scan image, or the corresponding data, and/or can output the measure of ballistic photons with a reduction of scattered photons.

The scan image can be generated using the adjusted photon detection counts for each of the detector's pixels (i.e., detector cells 31). In embodiments where the scanning technique corresponds to a computed tomography scan, a computed tomography image processor may be required to generate the image, by stitching together multiple 1D or 2D scan images (e.g., taken from various positions relative to the scanned object) to generate 2D or 3D absorption images or by using (e.g., by summing or averaging the adjusted counts corresponding to each detector's pixel) a collection of adjusted photon detection counts of multiple scans (acquisitions). In some embodiments, the generated scan image (i.e., absorption image) can be a 2D image or a 3D image or even a 4D image (e.g., comprising color coding of additional properties of the scanned volume, such as the type of material for example).

Note that the adjusted photon detection counts, that can correspond to a measure of ballistic photons with a reduction in the measure of scattered photons, can be determined by evaluating and removing a count/proportion of detected scattered photons from at least one shape parameter of the associated TPSF (e.g., slope of the TPSF, mean values, standard deviation total number of detected photons, full width at half maximum, maximum number of detected photons, timestamp of this maximum number, range of non-zero values, a shape parameter of a convolution/deconvolution, a shape parameter of a fit on part of the TPSF, or a combination thereof).

The contribution of detected scattered photons 36 in X-ray imaging can generally degrade the contrast-to-noise ratio (CNR), can reduce the accuracy of the attenuation measurements, and can generate cupping and streak artifacts. While it is present in virtually all radiography and computed tomography (CT) implementations, it is especially prevalent in systems in which a large volume is irradiated, such as in cone-beam CT or abdomen radiography. In the X-ray energy range for clinical applications (20-120 keV), absorption and scattering are almost equally likely. Thus, in abdomen imaging, the scatter contribution on individual pixels, measured as the ratio of detected scattered photons to the number of detected primary (or ballistic) photons (aka the scatter to primary ratio or SPR) can reach well over 100% as indicated by Siewerdsen et al. (DOI: 10.1118/1.1380430). The adverse effects of scatter contribution on CNR are generally compensated, again, with an increased radiation exposure.

One of the main advantages of the method described herein may be the reduction of uncertainties of ballistic photon counts and the increase of image quality for ToF computed tomography apparatuses. The method can allow to estimate the proportion of noise (i.e., non-ballistic X-photons), including scattered X-photons, in the measurement signal of a ToF computed tomography acquisition (count of detected X-photons as a function of the time of flight) or abdomen radiography.

The proposed method can allow to significantly improve the quality of the absorption image while significantly reducing the required time/temporal resolution of the X-ray detector of an X-ray imaging system or apparatus when used as an X-ray detector for ToF based removal of non-ballistic photons (e.g., scattered photons) by providing a significantly improved (i.e., more accurate) estimation of the number of measured ballistics photons from all measured photons. In an embodiment, this can comprise: measuring the instrument response function (IRF) for each detector pixel with an acquisition without any object between the source and the detector; scanning a subject-of-interest to measure its corresponding TPSF of photon impinging on each pixel; generating a TPSF dataset; correct the TPSF dataset with the IRF dataset using a temporal deconvolution to yield the estimated ToF distribution; estimate the proportion of ballistic photons from the estimated ToF distribution; and correct the final counts of detected ballistic photons by selecting a corresponding proportion from the total number/quantity of photons detected with the detector (e.g., for each detector cell).

The proposed method can be used as an alternative to anti-scatter grids, which can therefore allow to maintain a lower required dose of X-rays needed to produce a functional absorption image of the scanned object without its efficiency loss and diffusion creation in the grid material. This can be enabled since the proposed method can use ToF measurements to identify scattered photons and selectively exclude these identified scattered photons from the data of measured photons (i.e., adjusting photon detection count considered for generating the absorption image by evaluating a count/proportion of detected photons associated with scattered photons) used to reconstruct the absorption image. If the total temporal resolution is low enough to completely mitigate the effect of scatter noise, it could be possible to divide the dose by four in a cone beam arrangement. With a time resolution of around 100 picoseconds, the expected dose reduction for an abdomen scan is over 50%.

To this day, this has prevented the use of ToF measurements for scatter removal in X-ray imaging, since the temporal uncertainties of state-of-the-art X-ray imaging apparatuses can be of about 200 to about 300 picoseconds due to a combination of uncertainties or temporal resolutions of the X-ray source (e.g., time distribution of the X-ray pulse), the detector, the electronic components, etc. Evidently, these temporal uncertainties are reflected in the measurements of the ToF of the detected photons, which can therefore have equivalent uncertainties, in some cases, of over 300 picoseconds.

The method proposed herein can allow to estimate the number of ballistic photons based on the ToF measurements of all the detected photons rather than uniquely identifying scattered photons based on a rigid time threshold. This estimation can be accurate even with an uncertainty of over 300 picoseconds on the ToF measurement. This approach can significantly reduce the technological requirements (e.g., time resolution) for a ToF-based X-ray imaging system, thus significantly reducing the cost and allowing commercial usage of ToF analysis and imaging, even with typical and most widespread off-the-shelf electronics.

Some embodiments of the proposed method may consist of two main steps. First, an estimation of the scatter contribution using either a model or measurements. Second, a correction step that can modify the measured data to account for this estimated scatter contribution. Together, scatter rejection and correction can partly restore the CNR and reduce artifacts, but scatter contributions may still limit image quality when imaging large volumes.

The approach to solving the aforementioned problems may comprise measuring the ToF of the photons to form a histogram of the ToF (e.g., a number of detected photons for various intervals of ToF), i.e., the TPSF. The resulting dataset of the TPSF can then be used to estimate the number of ballistic photons detected which may then be used to generate the X-ray-based absorption image.

ToF is defined as the length of time between the photon's emission by the X-ray source and its arrival in the detection system where it is transduced into an electrical signal.

In the X-ray wavelength (or energy) range, the distance traveled by a photon is directly related to its ToF since the index of refraction is close to 1 regardless of the material and thus, all photons have a speed close to the speed of light in a vacuum. Measuring ToF requires knowing when the photons leave the source and when they arrive in the detection system. A way to create such a condition can be to use ultra-short pulses of photons to obtain a precise time of emission, and to measure the time of arrival of each individual photon using a time-correlated single photon counting detector.

The TPSF of a detector cell is defined as the distribution of the ToF of every photon that are detected. The TPSF can be obtained by classifying each detected photon in a histogram based on their ToF.

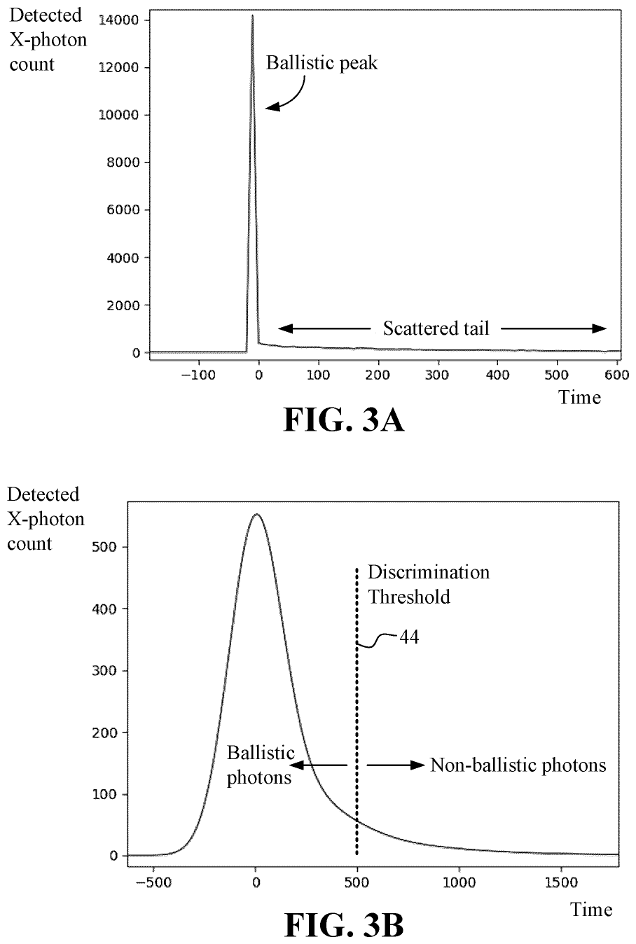

FIG. 3A shows an exemplary time-of-flight distribution (ToFD) of an X-ray imaging that can comprise a superposition of a first distribution of the ballistic photons, which all have more or less the same ToF associated with the peak around 0 seconds, with this distribution being alike a delta function, and a second distribution of the scattered photons, which can have a wide range of various times-of-flight associated with the small slowly decreasing tale between 0 seconds and about 500 picoseconds in this embodiment.

In a theoretical system with perfect time resolution, the distribution should have a shape resembling or having characteristics of (look like) a tall, very narrow peak representing ballistic photons followed by a long tail of scattered photons. In such a case, the ratio between the peak and the tail corresponds to the correction to be applied to the numbers of photons detected to obtain the true number of ballistic photons which are useful for obtaining the image with minimal noise. However, in practice, the limited temporal resolution of the detectors can result in a widening of the peak (e.g., peak of ballistic photons) and the tail (e.g., tail of scattered photons) of the TPSF by partially mixing them. Since the time resolution of the scanning system can be characterized and measured, it can be removed using mathematical methods, such as deconvolution, to recover an accurate estimate of the true ToFD with reduced temporal uncertainty.

The proportion of scattered photons from the ToFD (e.g., distribution over time of measured/detected photons, as a function of ToF) can be estimated using a mathematical model. The estimated proportion of scattered photons can then be used to adjust, correct or select an accurate or exact number/proportion of photons detected from this estimate.

It will be appreciated that the simplicity of the method proposed herein may be of particular interest and be significantly useful when using any ToF-based devices (e.g., ToF-based pulsed x-ray imaging as described in PCT publication WO2020/093140), since it can be used to reduce/relax the temporal resolution requirements required to obtain an image of similar quality generated using currently known (without using the TPSF) methods.

The differences of ToF between ballistic photons may be associated with the effect of spatial uncertainty on the exact location of emission and detection, as illustrated in FIG. 2B and which can generally be negligible. For instance, X-ray sources generally have a focal spot (the location that emits X-ray) smaller than 1 mm² which contributes to a maximum uncertainty of a ballistic photon ToF of 3.3 picoseconds. A second contribution may be the effect of scattered photons that can act similarly to a decreasing exponential function, its exact shape is dependent on the geometry of the imaged subject. If infinitely precise ToF measurements were done (i.e., theoretical embodiment), it would be easy to separate both contributions as the ballistic photon's contribution can be an extremely narrow delta function. This is the rationale behind previous work on time-of-flight X-ray imaging (e.g., PCT publication WO 2020/093140 A1). However, in an empirical setting, the ToFD can be affected by the time distribution of a pulse of emitted X-photons (source's pulse width) and/or by the precision (e.g., time resolution) of the detector. FIG. 3B presents an example of a ToFD, where the previously mentioned uncertainty sources/parameters have negatively affected (e.g., widened) the distribution, which can therefore cause both contributions (ballistic and scattered) to overlap significantly, making them hard to discriminate and can render them indistinguishable.

In the previously proposed time-of-flight computed tomography (e.g., PCT publication WO 2020/093140 A1), a discrimination threshold 44 or equivalents can be used to discriminate between ballistic photons, that can have a lower associated ToF (e.g., left side of the discrimination threshold 44), and the non-ballistic photons such as scattered photons that may have higher ToF (e.g., right side of the discrimination threshold 44). In preferred embodiments of the conventional method, this discrimination threshold can be determined and defined by a predetermined ToF value, which may be associated to an averaged maximum ToF for ballistic X-photons.

This widening effect can be represented mathematically by the convolution of the ToFD and the instrument response function (IRF) as in the following:

$$T o FD * IRF = T P S F ,$$

where * is the convolution operator.

The instrument response function also known as the impulse response is the instrument's temporal response, to a delta function that would be emitted by the source, of the X-ray imaging apparatus and more precisely of its various components without any object (e.g., time response calibration). The two main contributors to the instrument response function are the X-ray pulse width and the random timing jitter of the detector, which may also be convolved together:

$${W}_{p} * {R}_{D} \approx IRF ,$$

where Wₚ is the pulse width of the X-ray source and R_{D} is the random timing jitter of the detector. Other contributions to the instrument response function can include the mechanical uncertainties that can be associated with the source's focal spot, any misalignment of the detector, any random jitters on the source's trigger and any delays (e.g., a shift, in time, of the TPSF) that may be associated with a time response of any of the components of the imaging system. In some embodiments, these uncertainty contributions may generally be negligible.

In real X-ray imaging systems, with an empirical finite time precision (time resolution), the instrument response function can significantly hinder the capacity of identifying scattered photons and discriminate them from the ballistic photons. While an instrument response function can have a full width at half maximum (FWHM) of about 10 picoseconds or less for identifying almost all scattered photons, an instrument response function of about 300 picoseconds FWHM may only allow to identify 50% of scattered photons in a heavy scattering environment, such as the human abdomen. This can yield an improvement of the contrast-to-noise ratio (CNR) ten times lower than with an instrument response function having a FWHM of about 10 picoseconds. In scans with less scattering, a system with such an instrument response function may be unable to identify a significant part of scattered photons and thus does not improve the contrast-to-noise ratio.

Since it is difficult to accurately identify scattered photons in systems with large instrument response function, the proposed invention can use the TPSF directly to estimate the amount of detected ballistic photons regardless of which exact photon has scattered or not. This can be notably done by first measuring the instrument response function, and then deconvolving the TPSF by the instrument response function to provide an estimated time-of-flight distribution (EToFD). The IRF can either be measured or estimated. The instrument response function may typically be measured by completing an acquisition with nothing between the source and the detector. The instrument response function can correspond to the output of the system for a delta function. Since ballistic photons may be the only detected photons, which can have a ToFD that can act like a delta function, the measured distribution can be considered as the instrument response function. The instrument response function can also be estimated based on the expected pulse width and expected random jitters from the detectors. In some embodiments, this approach may result in a less accurate instrument response function measurement.

It will be appreciated that, for some embodiments, the exact shape of the ToFD may not be crucial, since the ratio between the initial peak of ballistic photons and the tail of scattered photons can be the most important characteristic when it comes to estimating the contribution of the non-ballistic photons (e.g., scattered photons) to the count of ballistic photons considered to generate the absorption image. In fact, when it comes to discriminating between ballistic and non-ballistic photons, an accurate estimate of the ToFD may be sufficient. Therefore, simpler algorithms may be used.

In some embodiments, the noise of the measured TPSF can then be removed since deconvolution can be an ill-posed problem. This can notably be done by first doing an Anscombe transformation on the TPSF, completing a wavelet denoising and finally by determining an inverse Anscombe transformation.

In some embodiments, the same denoising methodology can also be applied to a measured IRF. The deconvolution of the TPSF dataset by the instrument response function dataset can be computed using several methods. One such method is by minimizing the following function:

$${‖ Ax - b ‖}_{2}^{2} ,$$

where, A can be a lower triangular matrix whose elements are the instrument response function values, x can be a vector containing the current estimation for the ToFD and b can be a vector containing the TPSF data. The initial guess, which may highly impact the output of the minimization, can assume that every photon is ballistic and may therefore assume that the ToFD is a delta function multiplied by the sum of the TPSF.

Once the ToFD or EToFD is computed, it can be used to estimate the number (count) of detected ballistic photons. In some embodiments, this can be achieved by first identifying the maximum of the EToFD which can correspond to the ballistic contribution before integrating it. The integration bounds may be selected to include the range of values that form the “peak” of the distribution. For example, it may include, in one embodiment, the values contiguous to the maximum that are larger than a tenth of the maximum of the EToFD. This can a preferable alternative to applying, as per the standard (prior art) method, a simple discrimination threshold 44 that can require tighter/stricter temporal requirement (shorter X-ray pulse and detector with increased temporal resolution), by instead considering the proportion of the total count of detected photons.

Neural networks, also known as artificial intelligence, can be used as a good estimation tool, especially from nonlinear data involving convolution. The neural network may be programmed into a processor or any computing device that may be part of, integrated to/into, or coupled to/into, a separate device connected to the X-ray apparatus. In some embodiments, the neural network can be trained to use the shape (at least one shape parameter) of the TPSF in order to estimate the number of ballistic photons considered to generate the X-ray image.

It will be appreciated that these are only some of the possible methods that can be used to estimate the count of ballistic photons. In some embodiments, part of the distribution may be summed (integrated) or the value of the peak may be used, for example. The method to be used may be chosen as a function of how the EToD is generated.

Estimation and correction of the count of detected scattered photons using the proposed method that can utilize an iterative minimization deconvolution method shows great potential to allow better restoration of the CNR than the previously proposed time-of-flight scatter rejection method (e.g., as proposed in the PCT publication WO 2020/093140 A1), especially for apparatuses having timing-jitters of more than about 100 picoseconds. This new approach can allow to significantly reduce the time resolution requirement for the detector (e.g., requirements for time-of-flight computed tomography detectors). With state-of-the-art detectors, it should be possible to remove most of the adverse effects of scattered radiation on the CNR with the proposed method. Although it is not required for all embodiments of the proposed method, it will be appreciated that the instrument response function can include both the source pulse width and detector jitter as they can have a similar significant impact on the measurement.

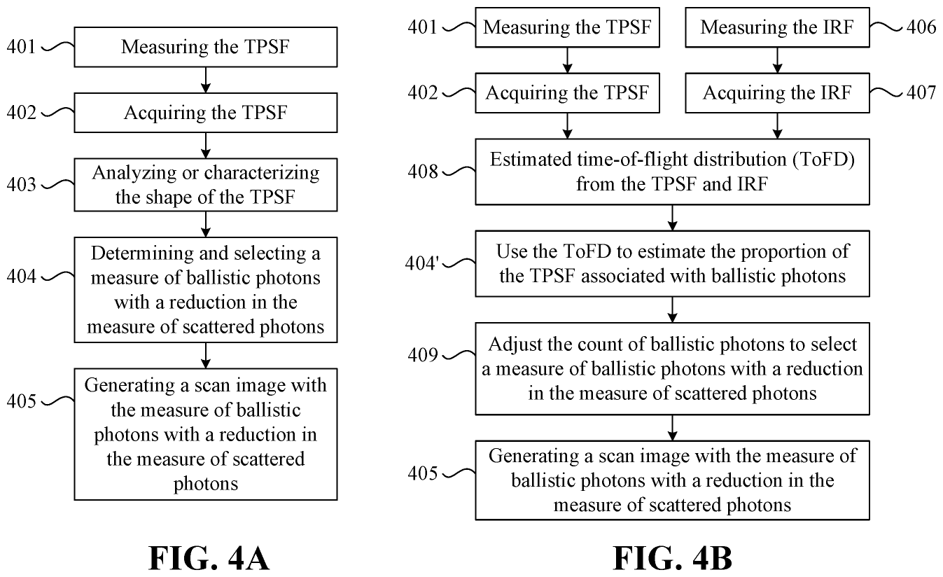

FIG. 4A presents a block diagram of the steps of a possible embodiment of the proposed method. A first step 401 may be required to complete an X-ray scan and acquire the ToF measurements of photons detected by an embodiment of a computed tomography apparatus, for example. Step 402 may be a following step if the TPSF is generated from the measurement data or can, in some embodiments, be a first step comprising acquiring a TPSF of a previously completed measurement, which may be extracted from some electronic system's memory. The following step 403 can comprise analyzing the distribution (shape) of the TPSF to characterize it. It will be appreciated that, in the present disclosure, the shape of the TPSF relates to the collection of all characteristics of the distribution of the TPSF and it is understood that these characteristics or collection of all characteristics (i.e., the shape) of the TPSF may be comprised (explicitly, implicitly, vaguely, cryptically, etc.) in the associated data, namely the TPSF dataset, which may be acquired with the detector of the imaging apparatus or extracted from a database or memory. In an embodiment, the following step 404 can comprise using the characteristics of a characterized TPSF to select the portion of the count of detected photons that should be corresponding to the ballistic photons (i.e., discarding the portion that should correspond to non-ballistic photons) to determine a measurement of ballistic photons with a reduction of scattered photons. For example, the identified characteristics of the shape of the TPSF may be used to find, within a lookup table (e.g., associated with a given imaging apparatus), the proportion to consider. An optional step 405 can be completed to use various counts/measurements of ballistic photons (e.g., for each detector's pixel) with a reduction of scattered photons of a given acquisition to generate a scan image that may be an X-ray absorption image.

The X-ray image can be generated from a CPU, a GPU, an FPGA or a combination thereof. It will be appreciated that the X-ray image can be generated using state of the art or conventional components of imaging apparatus of the current prior art or to be developed. The imaging can be performed with a computed tomography imaging apparatus able to make time correlated single photon counting measurements. For X-ray radiography the image can normally be obtained directly from the counts of photons per detector pixel. The image generator function as normally observed in computed tomography, where the image can be reconstructed using any conventional reconstruction algorithm such as the filtered-back-projection (FBP) or the Feldkamp-Davis-Kress algorithm for cone-beam CT (FDK) or any analogous algorithm and programs. Such algorithms can be implemented as part of the reconstruction toolkit (RTK) and the astra-toolbox libraries. In some embodiments, the estimated count of ballistic photons can preferably be normalized to the number of emitted photons.

FIG. 4B presents a block diagram of the steps of a possible embodiment of the proposed method comprising some steps that are similar to the steps of the block diagram of FIG. 4A. This can include step 401 for measuring the TPSF's data, a step 402 to acquire or generate the TPSF, and a step 405 to generate a scan image, all of which can be performed as previously described. This embodiment can further comprise a step 406 for measuring the temporal resolution and uncertainties of the X-ray imaging apparatus, which be used to generate, in step 407, the function associated with it (e.g., generate the IRF). In some embodiment, step 407 can comprise retrieving or acquiring IRF of a previously completed measurement, which may be extracted from some electronic system's memory. In a following step 408, the TPSF can be temporally deconvolved by the temporal resolution function (e.g., IRF) to estimate a more accurate distribution of the detected photons (e.g., ToFD). Step 404′ can comprise using and/or processing the estimated ToFD or EToFD to estimate a portion of these distributions associated with ballistic photons. In step 409, this estimated portion of distributions can be used to adjust the considered measure (count) of ballistic photons with a reduction of scattered photons. This adjusted count can then be used, in step 405, to generate a scan image (e.g., absorption image).

While the proposed method can allow for a relaxation of the temporal resolution requirements of some components of the associated apparatus (e.g., for the time-sensitive detector), it will be appreciated that the X-ray source of the proposed method and associated apparatus may preferably meet the same requirements proposed in the PCT publication WO2020/093140 and therefore provide an X-ray pulse having the temporal requirement, e.g., the full width at half maximum (FWHM), less than about 500 picoseconds and preferably less than about 100 picoseconds. Furthermore, the rising edge of the X-ray pulse can be less than about 500 picoseconds and is preferably of less than about 150 picoseconds.

While the time resolution requirements of the X-ray pulses can preferably remain the values mentioned above, the proposed method can allow for a relaxation in the time resolution requirements of the time-sensitive detector of the X-ray imaging system to avoid the need for it to be lower than 20 picoseconds (requirements of the prior art for time-of-flight X-ray imaging), which can be of up to about 500 picoseconds and preferably up to about 200 picoseconds. Furthermore, in some embodiments, the proposed method can be most relevant and suitable for systems that comprise a time-sensitive detector that may have a time resolution of more than about 10 to about 20 picoseconds. It will be appreciated that, in some embodiments, a time resolution of the detector may be lower than about 20 picoseconds and may allow to discriminate ballistic photons from non-ballistic photons (e.g., scattered photons) with a fairly satisfactory level of precision, which can imply that the proposed method may not provide a significative improvement to the standard/direct ToF measurement methods (e.g., PCT publication WO2020/093140).

### Simulation Results

The following presents non-limiting simulation results of a GATE simulation of the radiography of a head and a torso realized to generate input data for an iterative deconvolution algorithm to evaluate its performance. It will be appreciated that the tools (e.g., calculations, equipment, algorithm, etc.) and values (e.g., dimensions, energies, ratios, times, delays, etc.) mentioned in the following section may not be limiting the scope and various alternative values and/or tools that may be used for alternative embodiments of the proposed invention.

The embodiment considered for this simulation yielded significant results. In fact, the proposed method can improve the contrast-to-noise ratio (CNR) by more than about 12% in a low scattering environment. More specifically, the embodiments considered for the simulation presented herein resulted in an increase of 11.9% of the CNR to reach a CNR of about 96.2% with a reduced scatter photon contribution and more accurate selection (count) of ballistic photons than was obtained with the proposed method (e.g., with an embodiment of the deconvolution approach of time-of-flight computed tomography method), which had initially a CNR of about 84.3% of the image with only ballistic photons (selected using the time-of-flight computed tomography method as described in the PCT publication WO 2020/093140 A1).

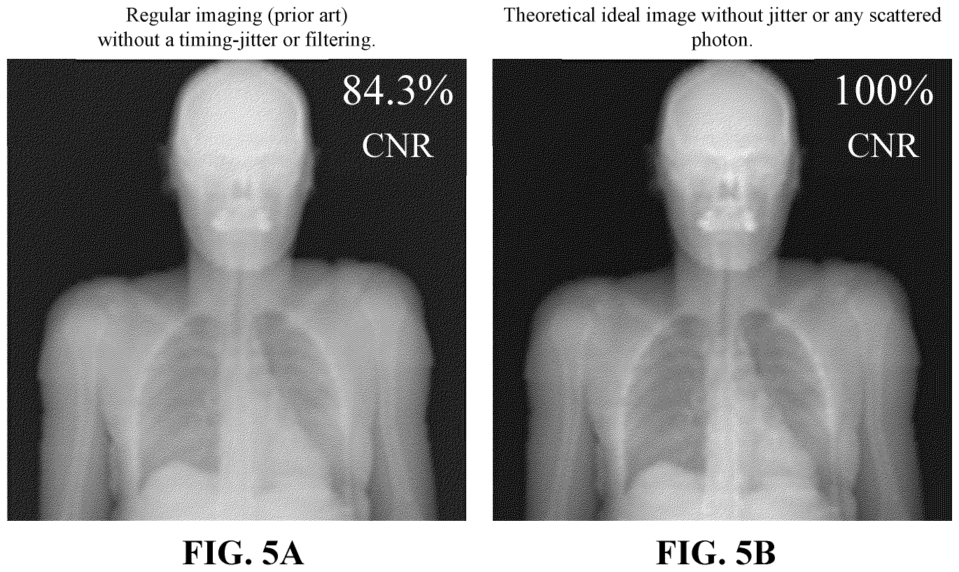

An embodiment of the simulation can comprise a GATE Monte Carlo simulator that can be used to simulate the X-ray radiography of a head and of a torso as illustrated in FIGS. 5A and 5B. The simulated system can comprise a point source emitting a monochromatic cone beam of 100 keV photons and a flat panel detector. All photons can be emitted simultaneously. The detector can be a 720×720×2.5 mm³ block of LYSO that may be placed 60 cm behind the phantom. Photons which deposit an energy lower than 20 keV in the detector can be discarded. During post-processing, photons may be placed according to their position in a 240×240 grid of pixels. The maximum expected ToF for ballistic photons can be removed from the simulated time of flight. Thus, a photon with a positive time of flight may arrive later than expected for ballistic photons regardless of the position in the detector. Then, a histogram of photon counts as a function of ToF can be generated for each pixel with 500 bins of 10 picoseconds. A Gaussian timing jitter may be applied to the histogram afterward using a convolution to evaluate multiple timing-jitters with a single simulation.

The histogram with the applied timing jitter can be used as an input to a function to be minimized. In one embodiment, the histogram with the applied timing jitter can be used as an input to a minimization process, which can minimize the function:

$${‖ Ax - b ‖}_{2}^{2} ,$$

previously introduced. The initial guess, which can highly impact the minimization result, may assume that every photon is ballistic and therefore should all have the same time of flight. The number (count) of ballistic photons can then be estimated by summing the negative part of the estimated ToFD. In order to demonstrate the efficiency of the proposed method in an empirical environment (with realistic conditions) a realistic (imperfect) time resolution for both the X-ray source and the detector of the X-ray imaging apparatus can be considered by adding a timing-jitter to the simulation. Therefore, this technique was simulated using a 300 and 100 picoseconds FWHM Gaussian timing-jitter.

In some of the simulations performed, the instrument response function or impulse response was simulated to resemble an IRF of a realistic time-of-flight computed tomography imaging apparatus.

As dictated by an embodiment of the proposed method, an accurate estimate of the true ToFD of the simulated measurement signal of detected X-photons having a TPSF can be retrieved using a deconvolution algorithm, which has been shown to enable accurate estimates of the ToFD, via iterative minimization and/or Tikhonov regularization (DOI: 10.1364/OL.37.002358).

The contrast-to-noise ratio can be computed by subtracting the mean value of a region in the lung between two ribs, for example, from the mean value of a region in the sternum, for example, divided by the standard deviation of the attenuation of the same lung region.

FIGS. 5A and 5B show the simulated images with all photons and with only the ballistic photons, respectively. In these results of a simulated embodiment, the CNR in the lower sternum region is degraded by 15.7% in the image with scattered photons. It will be appreciated that a CNR degradation can also be seen in the skull and along the ribcage.

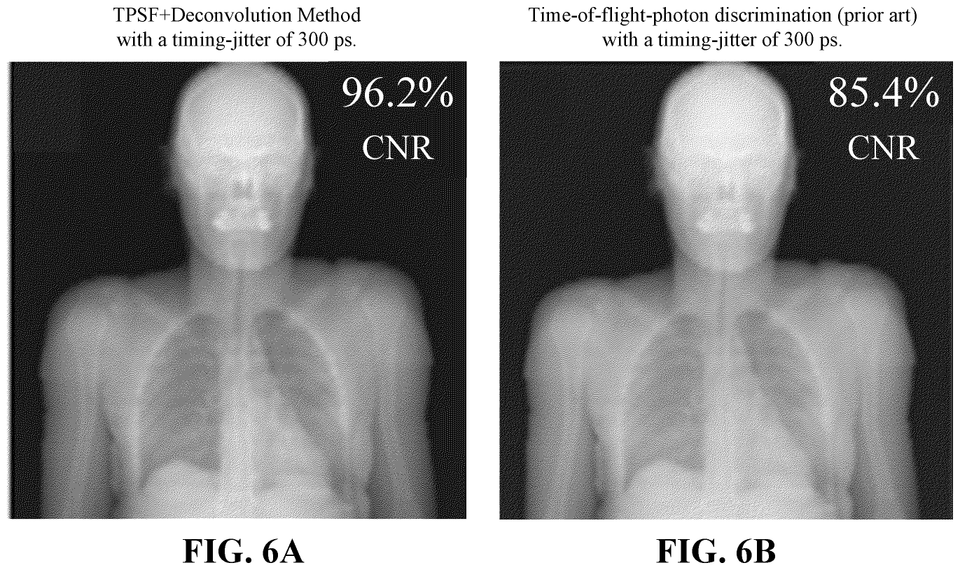

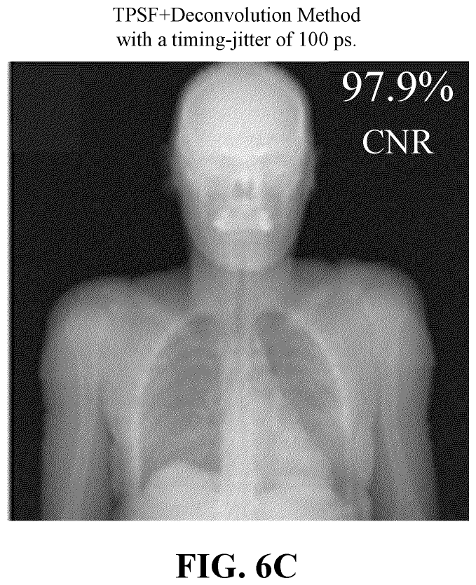

The proposed method that can generate improved scatter corrected images using the deconvolution algorithm, seen in the simulation results of FIG. 6A, can allow to restore a CNR up to about 96.2% of the original CNR when 300 picoseconds time resolution (jitter) is applied. FIG. 6B shows, for comparison, simulation results with the traditional ToF algorithm which is able to restore the contrast to only about 85.4% of the original also with a 300 picoseconds jitter, which is similar to the original CNR (with no rejection) of about 84.3% of the original. The proposed method can therefore improve the CNR by more than about 10%. This can be expected since, with poor timing jitter, the traditional method usually performs better to restoring a highly degraded image and has generally failed to correct a small CNR degradation. As shown in the simulation results of FIG. 6C, with a timing-jitter of 100 picoseconds, the deconvolution scatter correction algorithm can reach 97.9% of the original image's CNR.

### Time-of-Flight X-Ray Imaging

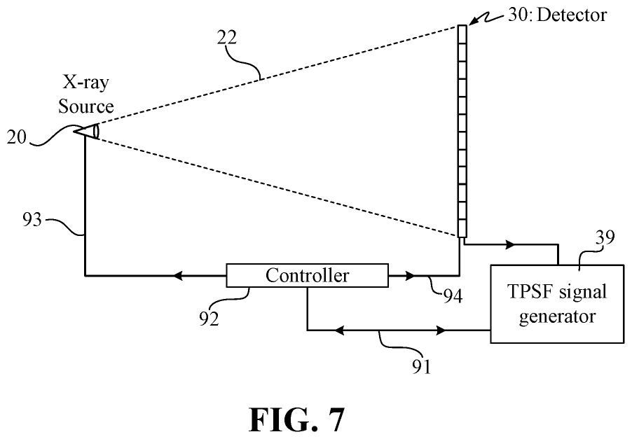

Some embodiments of the proposed apparatus, as illustrated in FIG. 7, can comprise a TPSF signal generator 39 that can be configured and used to send and/or to receive control signals 91 (e.g., control instructions, control data, etc.) to a controller 92 that can send a trigger signal 93 to an X-ray source 20 for generating, at a given time, a pulse of X-photons 22 (X-ray beam) and can send simultaneous or delayed detector control signals 94 to activate/deactivate the photodetector, or vice versa, to a time-sensitive detector 30 for measuring/detecting the source's X photons. In an embodiment, the control signal 91, sent from the controller 92 to the TPSF signal generator 39, can comprise a trigger signal 93 and/or detector control signal 94 or information about these signals. In an embodiment, the TPSF signal generator 39 can act as the controller 92.

In some embodiments, TPSF signal generator 39 can comprise circuitry for generating a TPSF signal dataset, which can include data/information about the detected photons such as their position on the detector array and their ToF (e.g., timestamp) or can count a number of detected photons for various ToF for each detector's pixels. In an embodiment, the TPSF signal dataset can be a time-dependent X photon detection signal that may be sent to a memory unit, an alternative circuitry (e.g., processor), an alternative apparatus to be further analyzed and processed, or a combination thereof.

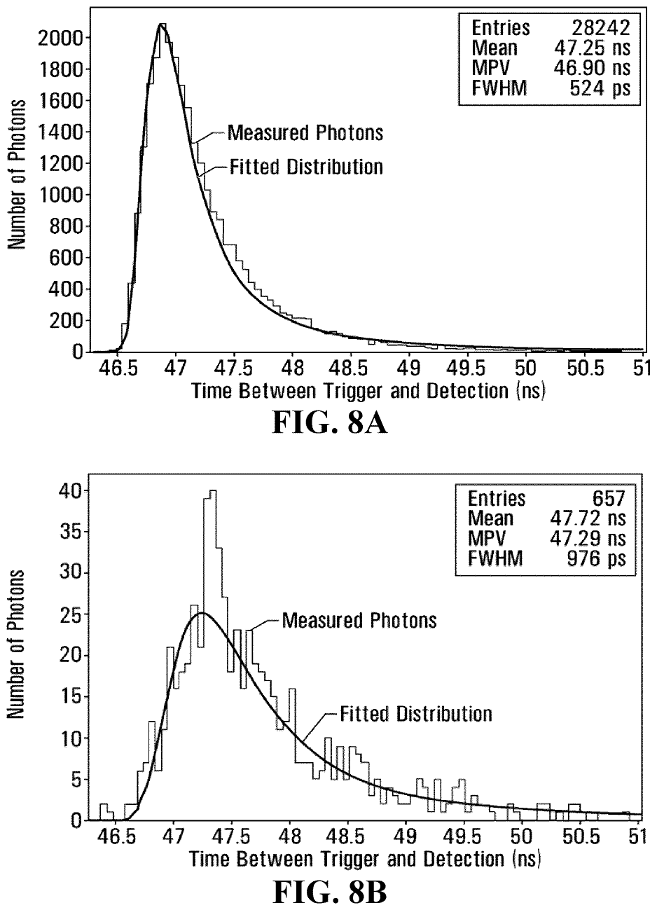

As presented in PCT publication WO2020/093140, an experiment was conducted to confirm the feasibility of observing ToF differences between scattered and transmitted (ballistic) photons. In this exemplary experiment, a 3×3 mm² silicon photomultiplier (SiPM) covered by a ˜500 μm thick lutetium yttrium oxyorthosilicate (LYSO) crystal was placed about 38 cm in front of a pulsed X-ray source with a mean photon energy of about 15 keV and a pulse width of about 60 picoseconds. Two measurements were made: one with nothing between the source and the detector and one with a ˜40 mm thick aluminum beam blocker. FIG. 8A shows the time between trigger and detection (TPSF) without the blocker, while FIG. 8B shows the time between trigger and detection with the blocker. In this experiment, 657 photons were detected after an acquisition of 36 hours. Once fitted with a Landau distribution, the most probable value (MPV) of the ToF with the beam blocker can be of about 390 picoseconds later than the MPV without the beam blocker, which may correspond to an increase of travel path of about 12 cm. This fits with the expected increase of travel path needed to circle the beam blocker by scattering on the X-ray enclosure. No ToF correction was made for the energy of the detected photons. Higher energy photons were detected earlier owing to their faster slope and better contrast-to-noise ratio, however, the increased ToF of scattered photons is observed at all energy levels at around 400 picoseconds. Dark counts and double detections were removed from both measurements.

An X-ray source is responsible for emitting X-rays. A source that emits X photons of only one energy is called monochromatic, otherwise it is called polychromatic (this terminology is in analogy with visible photons for which different energies correspond to different colors-chroma).

One of the possible means for producing X-rays can be by bending radially a beam of electrons (i.e., when the electrons accelerate perpendicular to their velocity) in synchrotrons using bending magnets, for example. In conventional X-ray tubes, the stream of X photons is continuous, but can be, very short pulses (or bursts) of X-rays of the order of at most about tens of picoseconds. Such short X-ray pulses can be generated via X-ray emission from femtosecond laser-produced plasmas on solid surfaces, for example. Another approach can be through the generation of high-order harmonics in gases which resorts to intense ultra-short laser pulses, which can be carried out in gas-filled hollow fibers. It will be appreciated that these approaches can be foreseen to be amenable to reasonable sizes for integration in medical imaging devices since ultra-short pulse laser technology is nowadays highly compact.

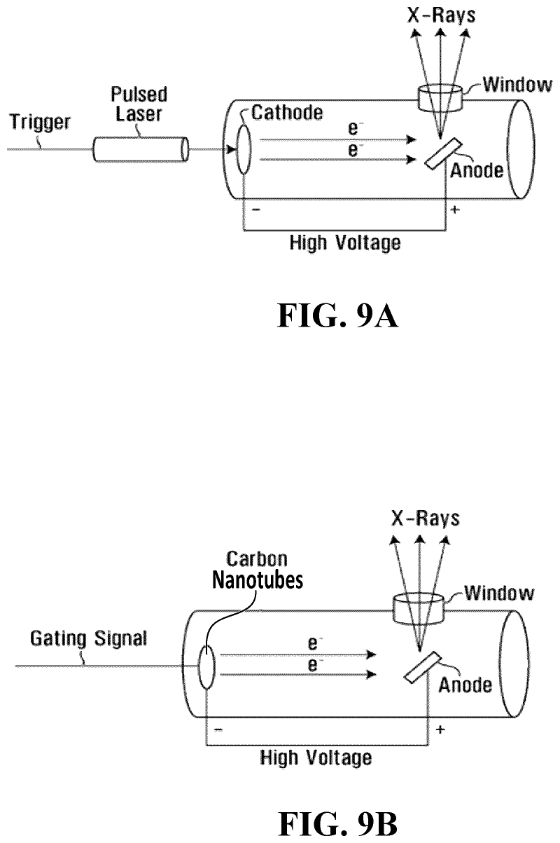

Another approach to generate ultra-short X-ray pulses can be one that has been developed for fluorescence lifetime measurements, whereby fluorescence is induced by X-ray excitation, where a pulsed laser diode emitting short pulses of light (e.g., less than about 100 picoseconds full width at half maximum (FWHM)) are directed onto a light-sensitive photocathode that emits short bursts of electrons with each light pulse impinging onto it, as schematically illustrated in FIG. 9A. The electrons are then accelerated towards an anode as in conventional X-ray tubes described above. Yet another approach to generate short X-ray pulses is to use an X-ray tube in which the electron beam can be very rapidly deflected as in a streak camera, with an electric pulsed field in such a way that it strikes the anode for a very short time interval in which bremsstrahlung X-rays can be generated.

Multiple implementations of the X-ray source, including the embodiments described herein, can generate suitably short pulses of X photons to reduce dosage while having a short enough rise time in intensity to allow for selection of ballistic photons using the method proposed herein and correspondingly provide the improvement in contrast of the resulting X-ray image. As an example, a trigger could be used to generate an ultra-short laser pulse directed onto the photocathode of an X-ray tube, generating a pulse of electrons accelerated in the tube with an electrical field. Similarly, an electron gun or canon could generate a continuous flow of electrons deflected or not on the X-ray emitter target. When the accelerated electrons hit the target then an X-ray pulse is generated towards the volume of interest using an aperture to form a fan or cone-shaped beam.

Another solution may be to generate X-ray pulses by replacing the photocathode with carbon nanotubes (CNT) as described in the prior art. The CNTs can be plated on top of the cathode as an electron emitter with the capability to be gated faster than the cathode alone directly with an electric signal (FIG. 9B) and operating at lower temperature.

It will be appreciated that different technology configurations can support embodiments of the invention and are not limited to the example described herein.

The proposed method can reduce the time resolution requirements of the detector, which is among the most important components to consider in the deployment of the proposed technology. There can be two main detection principles: direct conversion and indirect conversion. While the direct conversion of X photons in materials such as germanium or silicon is very attractive for high energy resolution, indirect conversion can be a preferred avenue thanks to its lower operating voltage and its proven better timing resolution. The use of a thin scintillator able to stop an X photon coupled to a high-speed photodetector such as a silicon photomultiplier (SiPM) or all its digital derivatives may be a good candidate for a complete system with timing performance of about 200 picoseconds to about 1 nanoseconds.

Circuitry could be integrated in 2.5D or 3D electronics along with the photodetector or located remotely outside the scanner. However, this approach will require a large data bandwidth and other approaches can be used.

In order to reduce the bandwidth, an adjustable and delayed trigger can be distributed in the scanner, so that the trigger can be self-adjusted from the center of the detector panel to the periphery or manually adjusted with programmable or fixed delay lines to take into account the source to flat panel distance variation from the center to the periphery. In some embodiments, the time-to-digital converter can take into account the position variation induced time variations at the detector.

Reducing the total time resolution requirements for the system, more specifically for the time-sensitive X-ray detector (i.e., not for the pulse width of the source), is one of the most important design aspects of the proposed method and apparatuses. The errors caused by each component of the system are added together in quadrature. Thus, reducing the pulse width of the source and the timing resolution of the detector cells can still be important to further increase the efficiency of the proposed method.

It will be appreciated that the use of the TPSF to approximate a more accurate ballistic photon count (i.e., with a reduction in the measure of scattered photons) can significantly reduce or relax the requirements for the timing resolution of the detectors. This can further allow for higher jitter between detector cells. The method of using of the TPSF to approximate a more accurate ballistic photon count can therefore be used to use time-of-flight X-ray imaging with a wider range of existing technologies and/or can be used to improve the quality of the image (e.g., contrast-to-noise ratio) of existing time-of-flight X-ray imaging systems.

Embodiments can be implemented in a variety of systems. The following fields have been identified as potential interesting applications of time-of-flight X-ray imaging (whether 2D or 3D):
Pediatric (where the dose of radiation can be reduced to acceptable levels);
Preclinical (where spatial resolution can be improved due to reduced contrast-to-noise ratio);
Dental care (where X-ray dose and the form factor are important);
Bariatric patients (where the contrast is a problem);
Extremities (where the form factor and the X-ray dose are significant criteria);
Interventional radiology (form factor, dose and resolution);
These systems may require different adjustments in their design options (or optimization) for an X-ray dose, spatial resolution, contrast and form factor as indicated in parentheses in the list of applications above. X-ray imaging (whether 2D or 3D) is rarely used for pediatric patients because of the high radiation dose associated with current X-ray imaging procedures. Since the method presented herein can be used with a relatively smaller dose can allow for extensive use of the technique for this application. Scans of extremities, dental care and interventional radiology can also be optimized for an X-ray dose to reduce the impact of the repeated use required by those applications.

Spatial resolution is particularly important for both pre-clinical and interventional radiology. A significantly higher spatial resolution can be obtained by using embodiments as described herein in conjunction with single photon avalanche diodes (SPADs) detectors to precisely pinpoint the location of the interaction of the X-ray with the detectors. Although SPADs can have better timings (lower timing jitter), they may not have a better interaction depth. Some embodiments can be used in conjunction with silicon photomultipliers (e.g., 1×1 mm²) which may be enough for the spatial resolution. The quality of the discrimination, along with the spatial resolution, can also be improved by using a magnification process such as increasing the distance between the volume of interest and the detector system.

Scanning bariatric patients requires photons of higher energies than the standard range of energy used in X-ray imaging (whether 2D or 3D) and yields a lower contrast image. Dynamic spatial reconstruction (DSR) is particularly useful for scanning bariatric patients since larger volumes generate more scatter noise which is easier to remove with our approach since the photons generally scatter more than once in the subject in those cases.

Finally, embodiments of the invention can be well suited timed or synchronized imaging where the X-ray scan could be completed according to an external signal such as respiratory gating or cardiac to avoid motion artifacts and better visualize the organ.

1. An X-ray imaging apparatus comprising:
a pulsed X-ray source having a control signal;
a time-sensitive X-ray detector having a time-dependent X-ray photon detection signal output;
circuitry, connected to said control signal and said time-dependent X-ray photon detection signal output, for collecting a temporal point spread function (TPSF) dataset from a time-dependent X-ray photon detection signal of said time-sensitive X-ray detector, wherein a TPSF of said TPSF dataset is equal to a time of flight distribution (ToFD) convolved with an instrument response function (IRF) of said X-ray imaging apparatus; and
a processor and memory storing program code that, when executed by said processor, causes said processor to estimate a number of ballistic photons based on at least one shape parameter of a TPSF of said TPSF dataset to provide a measure of ballistic photons with a reduction in scattered photons wherein the number of ballistic photons estimated corresponds to a ballistic portion of the ToFD.

2. (canceled)

3. The apparatus as defined in claim 1, wherein said estimating said number of ballistic photons is based on a distribution resulting from a temporal deconvolution of said TPSF by said IRF.

4. The apparatus as defined in claim 1, wherein said circuitry comprises said processor and said memory, and wherein said storing program code, when executed by said processor, causes said processor to perform said collecting of said TPSF dataset from said time-dependent X-ray photon detection signal.

5. The apparatus as defined in claim 1, further comprising an image processor and wherein said program code, when executed by said image processor, further causes said image processor to generate an X-ray image from said measure of ballistic photons with the reduction in scattered photons.

6. The apparatus as defined in claim 1, wherein a temporal resolution of said time-sensitive X-ray detector is more than about 20 picoseconds.

7. The apparatus as defined in claim 1, wherein said pulsed X-ray source is selected from the group consisting of:
said pulsed X-ray source comprising a pulsed laser source responsive to said control signal; and a photoelectric material arranged to receive a light pulse from said pulsed laser source and to emit a burst of electrons in response thereto; wherein electrodes are arranged to accelerate said burst of electrons;
said pulsed X-ray source comprising a high-voltage source, electrodes connected to said high-voltage source for accelerating electrons, and an X-ray emitting target material arranged to receive said electrons following acceleration by said electrodes so as to produce a pulse of X-rays; and
said pulsed X-ray source comprising deflection electrodes for steering said electrons accelerated by said electrodes connected to said high-voltage source to controllably hit said X-ray emitting target material.

8. (canceled)

9. (canceled)

10. (canceled)

11. (canceled)

12. The apparatus as defined in claim 1, wherein said time-sensitive X-ray detector is arranged with respect to said pulsed X-ray source so as to provide a different time of flight for said ballistic photons as a function of a pixel location within said time-sensitive X-ray detector.

13. The apparatus as defined in claim 1, wherein said time-sensitive X-ray detector is selected from the groups consisting of:
said time-sensitive X-ray detector comprising an X-ray sensitive scintillator and a light sensor array coupled with said scintillator for measuring X-ray detection events in said scintillator; and
said time-sensitive X-ray detector comprising an X-ray sensitive detector based on a direct conversion of photons to electrons for measuring X-ray events in said time-sensitive X-ray detector.

14. (canceled)

15. (canceled)

16. The apparatus as defined in claim 1, further comprising a motorized mounting for moving said pulsed X-ray source and said time-sensitive X-ray detector with respecting to an object or subject to be imaged, wherein said program code, when executed by said image processor, causes said image processor to provide a three-dimensional image.

17. The apparatus as defined in claim 1, wherein said pulsed X-ray source produces a cone beam of photons and said time-sensitive X-ray detector is arranged in a 2D array of pixels to detect at least some of said photons.

18. The apparatus as defined in claim 1, wherein a temporal uncertainty between a time of said control signal and a time of detection of an X-photon by said time-sensitive X-ray detector is greater than 200 picoseconds.

19. The apparatus as defined in claim 18, wherein an image generated from said measure of ballistic photons with the reduction of scattered photon have a contrast-to-noise ratio at least 8% higher than the contrast-to-noise ratio of an image generated from a measure of ballistic photons with a reduction of scattered photons having a time-of-flight higher than a predetermined time-of-flight.

20. The apparatus as defined in claim 1, wherein a rise time of a pulse emitted by said pulsed X-ray source is less than 0.15 nanoseconds.

21. A device for producing an X-ray image comprising:
a processor; and
memory storing program code;
wherein said program code, when executed by said processor, causes said processor to:
estimate, for each TPSF of a provided TPSF dataset, a number of ballistic photons based on at least one shape parameter of said TPSF to provide a measure of ballistic photons with a reduction in the measure of scattered photons, wherein said each TPSF is equal to a time of flight distribution (ToFD) convolved with an instrument response function (IRF) of said X-ray imaging apparatus, and wherein the number of ballistic photons estimated corresponds to a ballistic portion of the ToFD; and
generate an image from said measure of ballistic photons with the reduction in scattered photons.

22. The device as defined in claim 21, further comprising a computed tomography image processor and wherein said program code, when executed by said computed tomography image processor, causes said computed tomography image processor to generate an X-ray image from each of said measure of ballistic photons with the reduction in scattered photons.

23. The device as defined in claim 21, wherein said computed tomography image processor is said processor used for said estimating said number of ballistic photons.

24. The device as defined in claim 21, wherein said estimating said number of ballistic photons is based on a distribution resulting from a temporal deconvolution of said TPSF by said IRF.

25. A method for generating an X-ray image comprising:
providing an X-ray imaging apparatus comprising a pulsed X-ray source and a time-sensitive X-ray detector;
collecting a temporal point spread function (TPSF) dataset from a time-dependent X-ray photon detection signal of said time-sensitive X-ray detector, wherein said TPSF dataset comprises a TPSF for each one of a plurality of detector cells of said time-sensitive X-ray detector, said TPSF being equal to a time of flight distribution (ToFD) convolved with an instrument response function (IRF) of said X-ray imaging apparatus;
estimating a number of ballistic photons based on at least one shape parameter of one of a plurality of said TPSF comprised in said TPSF dataset to provide a measure of ballistic photons with a reduction in scattered photons, wherein the number of ballistic photons estimated corresponds to a ballistic portion of the ToFD;
providing an image processor; and
generating, with said image processor, the X-ray image from said measure of ballistic photons with the reduction in scattered photons.

26. The method as defined in claim 25, wherein said time-dependent X-ray photon detection signal comprises the time-dependent X-ray photon detection pixel signal of each detector cell of said time-sensitive X-ray detector.

27. The method as defined in claim 25, wherein said estimating said number of ballistic photons is based on a distribution resulting from a temporal deconvolution of said TPSF by said IRF.
